(19) United States
(12) Patent Application Publication
(10) Pub. No.: US 2008/0039916 A1
(43) Pub. Date: Feb. 14, 2008

(54) Distally distributed multi-electrode lead

(76) Inventors: Olivier Colliou, Los Gatos, CA (US); Marc Jensen, Los Gatos, CA (US)

Correspondence Address: BOZICEVIC, FIELD & FRANCIS LLP;(PROTEUS BIOMEDICAL, INC), 1900 UNIVERSITY AVENUE, SUITE 200, EAST PALO ALTO, CA, 94303, US

(21) Appl. No.: 11/890,281

(22) Filed: Aug. 2, 2007

Related U.S. Application Data
(60) Provisional application No. 60/821,803, filed on Aug. 8, 2006.

(51) Int. Cl. A61N 1/04

(52) U.S. Cl. 607/116

(57) ABSTRACT

Distally distributed multi-electrode leads are provided. The leads have a distally distributed electrode satellite which includes a processor and at least one individually addressable electrode positioned on the lead distal to the processor, e.g., in a tapered distal end of the lead. Also provided are implantable pulse generators that include the inventive leads, as well as systems and kits having components thereof, and methods of using the subject devices.

### CROSS-REFERENCE TO RELATED APPLICATIONS

Pursuant to 35 U.S.C. § 119 (e), this application claims priority to U.S. Provisional Application Ser. No. 60/821,803 filed Aug. 8, 2006; the disclosure of which priority application is herein incorporated by reference.

### INTRODUCTION

1. Field of the Invention

The present invention relates generally to implantable medical leads.

2. Background

Pacemakers and other implantable medical devices find wide-spread use in today's health care system. A typical pacemaker includes stimulating electrodes that are placed in contact with heart muscle, detection electrodes placed to detect movement of the heart muscle, and control circuitry for operating the stimulating electrodes based on signals received from the detection electrodes. Thus, the pacemaker can detect abnormal (e.g., irregular) movement and deliver electrical pulses to the heart to restore normal movement.

A common method for correcting arrhythmias of the heart is cardiac resynchronization therapy (CRT). Methods for CRT are well known. Most often, three leads are placed in the heart to sense cardiac activity and pace when needed. One lead is placed in the right ventricle, one in the atrium, and one lead is used to pace the left ventricle.

One of the current limitations in the practice of CRT is the difficulty in placing the lead for pacing of the left ventricle. There are a limited number of veins in the left ventricle, and the veins taper to a narrow diameter at the distal end. For the most part, the diameter of the vein at the distal end is smaller than the diameter of currently available implantable leads. This makes it very difficult to reach more distal parts of the vein with the lead. Because the optimal pacing location is often close to the distal end of the left ventricular veins, a physician is forced to advance the lead as far distally as possible.

There have been several approaches to address this issue. Stokes suggests a lead which achieves a small diameter by hardwiring a small number of electrodes over the length of the lead, and using a sheath of high tensile stiffness to stabilize the lead. (U.S. Pat. No. 6,366,819). This confers the advantage of allowing more than one lead to reside in the same vein.

Scheiner et al. teach making a lead with a tapered flexible tip for easier placement in the vein. They suggest electrodes located along the tapered portion and hard-wired back to a signal generator connector located at the proximal tip of the lead (U.S. Pat. No. 6,584,362).

The size of the processor, or control chip is one of the factors limiting the diameter of the lead. The current inventors have improved on current implantable lead designs by placing the hermetically sealed processor in the larger diameter portion of the lead, and hard-wiring it to multiple electrodes placed more distally, so that the lead can be tapered down to a smaller diameter. This allows the lead to be placed further down the vein than before, offering a wider range of possible pacing locations.

Another limitation in the practice of CRT is that following implantation of the lead, the rotational orientation of the electrodes can not be predetermined in many currently employed devices due to the tortuous nature of the vessels in the heart. As such, many currently employed lead devices employ cylindrical electrode designs that are conductive to tissue around the entire diameter of the lead. This ensures that some portion of the cylindrical electrode contacts excitable tissue when it is implanted. Despite the multiple devices in which cylindrical continuous ring electrodes are employed, there are disadvantages to such structures, including but not limited to: undesirable excitation of non-target tissue (e.g. the phrenic nerve), which can cause unwanted side effects, increased power use, etc.

An innovative way to address this problem is to employ segmented electrode structures, in which the circular band electrode is replaced by an electrode structure made up of two or more individually activatible and electrically isolated electrode structures that are configured in a discontinuous band. Such segmented electrode structures are disclosed in published PCT application Publication Nos. WO 2006/069322 and WO2006/029090; the disclosures of which are herein incorporated by reference.

The ability to provide stimulation through a segmented electrode array avoids the often traumatic problem of having to reposition a lead when the lead electrodes are in improper positions. For example, repositioning the lead may be necessary when the lead either does not provide adequate stimulation of cardiac tissue or the lead stimulates inappropriate tissues such as the phrenic nerve. With a segmented electrode array, a physician of ordinary skill is able to focus stimulation to the optimal pacing areas, and away from problematic pacing areas by activating or deactivating certain electrodes in the array without having to reposition the lead.

It would therefore be an important advancement in cardiology to have a single body lead that is able to reach more distal portions of the left ventricular veins, able to selectively excite a wide range of locations within the vein, and able to directionally focus the excitation pulses.

### SUMMARY

The present invention provides distally distributed multi-electrode leads. The leads include at least a distally distributed satellite electrode which includes a processor and at least one individually addressable electrode positioned on the lead distal to the processor, e.g., in a tapered distal end of the lead. Also provided are implantable pulse generators that include the inventive leads, as well as systems and kits having components thereof, and methods of using the subject devices.

### BRIEF DESCRIPTION OF THE FIGURES

FIG. 1 illustrates a lead with multi-directional electrodes on the proximal end, followed by a hermetically sealed chip hard wired to ring electrodes on a smaller diameter distal end, with a curved tip.

FIG. 2 illustrates a lead with the entire smaller diameter distal portion molded in a curved “S” shape.

FIG. 3 illustrates a lead with a gradually tapering distal end.

FIG. 4 illustrates a lead with four electrodes placed circumferentially at the smaller diameter distal end.

FIG. 4A is a cross-sectional view of the larger diameter proximal portion of the lead in FIG. 4, with four electrodes placed circumferentially around the control chip.

FIG. 4B is a cross-sectional view of the lead in FIG. 4 at the distal end, where the electrodes are located circumferentially around the lead axis, but do not have a control chip in the center.

FIG. 5A illustrates the expandable distal electrodes in a collapsed state.

FIG. 5B illustrates the expandable distal electrodes in an expanded state.

FIG. 5C is a closer view of the expandable distal electrodes in a collapsed state.

FIG. 5D is a closer view of the expandable distal electrodes in an expanded state.

### DETAILED DESCRIPTION

The present invention provides a distally distributed multi-electrode lead with a unique ability to reach regions of the heart which were previously unavailable, combined with the ability to deliver selective tissue activation without the need to reposition the lead. The distally distributed multi-electrode lead has a processor coupled to distally located electrodes, which allows the lead to taper to a smaller diameter distally, and in some embodiments includes one or more additional electrode satellites located proximally. Also provided are implantable pulse generators that include the leads, as well as systems and kits having components thereof, and methods of making and using the devices.

Distally Distributed Multi-Electrode Lead

Embodiments of the invention include implantable elongated flexible structures (e.g. leads) with distally distributed multi-electrode lead assemblies, which include one or more distally distributed satellite electrodes. The distally distributed satellite electrodes include a processor (i.e. integrated circuit, or control chip), and at least one individually addressable electrode coupled to the more proximally located processor. In some embodiments, the distally distributed satellite electrode is a segmented electrode, comprising two or more individually addressable electrodes located distal to the processor. In some embodiments, the implantable elongated flexible structure (e.g. lead) is a multi-electrode lead with at least one additional electrode satellite positioned proximal to one or more distally distributed satellite electrodes. In some embodiments, the at least one additional electrode satellite positioned proximal to one or more distally distributed satellite electrodes is a segmented electrode, comprising two or more individually addressable electrodes. In some embodiments, the distal end of the lead is tapered relative to the proximal end.

One or more of the distal processors can be hermetically sealed, and can be hard-wired to a plurality of electrodes located distally. In between the processor and the closest hard-wired electrode, the diameter of the implantable elongated flexible structure, or lead, can taper to a smaller diameter. The diameter of the distal end can be about 1 French to about 5 French, more specifically about 1.5 French to about 3 French, most specifically about 2 French. The tapered distal end can then maintain a constant diameter through the remaining length of the lead.

In one embodiment, at least one of the individually addressable electrodes of the distally distributed satellite electrode can be positioned in the tapered end. In another embodiment, two or more of the individually addressable electrodes can be positioned in the tapered end. The tapered distal portion of the lead can have a length of about 0.5 cm to about 10 cm, such as from about 1 cm to about 6 cm, and including from about 1.5 cm to about 3 cm.

In one embodiment of the distally distributed multi-electrode lead, the distal portion can be gradually tapered from the larger diameter of the proximal end, to a smaller diameter at the distal end. The control chip, or processor, of the distally distributed satellite electrode can be located proximal to the beginning of the tapered end, and therefore in some embodiments the processor of the distally distributed satellite electrode is not located in the tapered end. In other embodiments, the processor of the distally distributed satellite electrode can be located in the tapered distal end. The distal taper gives the advantage of conforming to the natural shape of the vein, which allows for optimal contact of the electrodes with the surrounding tissue. For instance, in the case of four ring electrodes located on the distal portion which are hard-wired to a proximal control chip, there will be four wires running to the location of the first electrode (with one connecting), three wires continuing to the next electrode, two wires to the third electrode, and one wire to the last electrode. Naturally, as there are fewer wires needed at the more distal points, the lead can taper accordingly.

The processor and the at least one individually addressable electrode of the distally distributed satellite electrode can be separated by a distance of 30 mm or less, such as 25 mm or less, and including 20 mm or less. In some embodiments, the processor and the at least one individually addressable electrode of the distally distributed satellite electrode can be separated by a distance of 1 mm or more, such as 5 mm or more, 10 mm or more, including 15 mm or more, or 20 mm or more. The processor can be located from 1 mm to 30 mm from the at least one individually addressable distal electrode coupled to it, such as from 10 mm to 20 mm, and including from 15 mm to 20 mm.

In further embodiments, the inventive implantable elongated flexible structures, e.g. leads, may include additional electrode satellite structures located proximally, which may be segmented electrodes. These electrode satellite structures may further include control circuitry, e.g., in the form of an integrated circuit, or IC (e.g., an IC inside of the support), such that the satellite structure is addressable. In certain embodiments, the structure includes two or more segmented electrode satellites, such as three or more segmented electrode satellites, including four or more segmented electrode satellites.

By segmented electrode structure is meant an electrode structure that includes two or more, e.g., three or more, including four or more, disparate electrode elements. Embodiments of segmented electrode structures are disclosed in Application Serial No.: PCT/US2005/031559 titled “Methods and Apparatus for Tissue Activation and Monitoring,” filed on Sep. 1, 2006; PCT/US2005/46811 titled “Implantable Addressable Segmented Electrodes” filed on Dec. 22, 2005; PCT/US2005/46815 titled “Implantable Hermetically Sealed Structures” filed on Dec. 22, 2005; Ser. No. 11/734,617 titled “High Phrenic, Low Pacing Capture Threshold Pacing Devices and Methods” filed on Apr. 12, 2007; and Ser. No. 11/777,981 titled “Focused Segmented Electrode”, filed on Jul. 13, 2007, the disclosures of the various segmented electrode structures of these applications being herein incorporated by reference.

In certain embodiments, the segmented electrodes are “addressable” electrode structures. Addressable electrode structures include structures having one or more electrode elements directly coupled to control circuitry, e.g., present on an integrated circuit (IC). Addressable electrodes include satellite structures that include one more electrode elements directly coupled to an IC and configured to be placed along a. Examples of addressable electrode structures that include an IC are disclosed in application Ser. No. 10/734,490 titled “Method and System for Monitoring and Treating Hemodynamic Parameters” filed on Dec. 11, 2003; PCT/US2005/031559 titled “Methods and Apparatus for Tissue Activation and Monitoring,” filed on Sep. 1, 2006; PCT/US2005/46811 titled “Implantable Addressable Segmented. Electrodes” filed on Dec. 22, 2005; PCT/US2005/46815 titled “Implantable Hermetically Sealed Structures” filed on Dec. 22, 2005; Ser. No. 11/734,617 titled “High Phrenic, Low Pacing Capture Threshold Pacing Devices and Methods” filed Apr. 12, 2007; and Ser. No. 11/777,981 titled “Focused Segmented Electrode”, filed on Jul. 13, 2007; the disclosures of the various addressable electrode structures of these applications being herein incorporated by reference. In these embodiments where an IC is present, the segmented electrode structure may include IC holding elements that immobilize an IC relative to the other elements of the structure.

As described above, the electrode satellite can have a plurality of individually addressable electrodes arranged circumferentially around a control chip, which can be hermetically sealed. The chips can be multiplexed, so that there can be only two wires running between each chip. However, the electrodes can be individually addressed. The chips, in turn, can individually address each of the electrodes located circumferential to them. The chip can control whether each electrode acts as a cathode, an anode, or is turned off, as well as vary the voltage applied. It can also configure the electrode for pacing, sensing, or another function. This allows the versatility of being able to change pacing locations without repositioning the lead, as well as allowing focused directional stimulation to capture only the desired tissue.

The segmented electrode structures may vary considerably, so long as the different electrode elements are sufficiently proximal to each other to generate the desired electric stimulation. Distances between the electrode structures may vary, where in certain embodiments, the distances are about 1000 μm or less, such as about 500 μm or less, and in certain embodiments range from about 5 μm to about 1000 μm, such as from about 50 μm to about 500 μm and including from about 100 to about 300 μm, e.g., about 200 μm.

Where the segmented electrode structure is present on an implantable elongated flexible structure, e.g. lead, or analogous carrier, the electrode structure may be conductively coupled to an elongated conductive member, e.g., to provide for communication with a remote structure, such as a remote controller, e.g., which may be present in a structure which is known in the art as a “can.” As such, in certain embodiments, the segmented electrode structures are electrically coupled to at least one elongated conductor, which elongated conductor may or may not be present in a lead, and may or may not in turn be electrically coupled to a control unit, e.g., that is present in a pacemaker can. In such embodiments, the combination of segmented electrode structure and elongated conductor may be referred to as a lead assembly.

In certain embodiments, each electrode element of the segmented structure may be coupled to its own conductive member or members, such that each electrode element is coupled to its own wire. In these embodiments the structure or carrier, e.g., lead, on which the structure is present may be torqueable, such that it can be turned during and upon placement of the lead so that upon activation, the electrode elements produce stimulation in the desired, focused direction.

In yet other embodiments, the electrode elements of the structure are present on a multiplex lead, such that two or more disparate electrode structures are coupled to the same lead or leads. A variety of multiplex lead formats are known in the art and may readily be adapted for use in the present devices. See e.g., U.S. Pat. Nos. 5,593,430; 5,999,848; 6,418,348; 6,421,567 and 6,473,653; the disclosures of which are herein incorporated by reference. Of particular interest are multiplex leads as disclosed in published U.S. Patent application no. 2004-0193021; the disclosure of which is herein incorporated by reference.

Of interest are structures that include an integrated circuit (IC) electrically coupled (so as to provide an electrical connection) to two or more electrode elements. The term “integrated circuit” (IC) is used herein to refer to a tiny complex of electronic components and their connections that is produced in or on a small slice of material, i.e., chip, such as a silicon chip. In certain embodiments, the IC is a multiplexing circuit, e.g., as disclosed in PCT Application No. PCT/US2005/031559 titled “Methods and Apparatus for Tissue Activation and Monitoring” and filed on Sep. 1, 2005; the disclosure of which is herein incorporated by reference. In the segmented electrode structures, the number of electrodes that is electrically coupled to the IC may vary, where in certain embodiments the number of 2 or more, e.g., 3 or more, 4 or more, etc., and in certain embodiments ranged from 2 to about 20, such as from about 3 to about 8, e.g., from about 4 to about 6. While being electrically coupled to the IC, the different electrodes of the structures are electrically isolated from each other, such that current cannot flow directly from one electrode to the other. In these embodiments, the lead need not be torqueable, since the desired focused stimulation can be achieved through selective activation of electrodes.

As summarized above, the invention provides implantable medical devices that include the electrode structures as described above. By implantable medical device is meant a device that is configured to be positioned on or in a living body, where in certain embodiments the implantable medical device is configured to be implanted in a living body. Embodiments of the implantable devices are configured to maintain functionality when present in a physiological environment, including a high salt, high humidity environment found inside of a body, for 2 or more days, such as about 1 week or longer, about 4 weeks or longer, about 6 months or longer, about 1 year or longer, e.g., about 5 years or longer. In certain embodiments, the implantable devices are configured to maintain functionality when implanted at a physiological site for a period ranging from about 1 to about 80 years or longer, such as from about 5 to about 70 years or longer, and including for a period ranging from about 10 to about 50 years or longer. The dimensions of the implantable medical devices of the invention may vary. However, because the implantable medical devices are implantable, the dimensions of certain embodiments of the devices are not so big such that the device cannot be positioned in an adult human.

Embodiments of the invention also include medical carriers that include one or more electrode assemblies, e.g., as described above. Carriers of interest include, but are not limited to, vascular lead structures, where such structures are generally dimensioned to be implantable and are fabricated from a physiologically compatible material. With respect to vascular leads, a variety of different vascular lead configurations may be employed, where the vascular lead in certain embodiments is an elongated tubular, e.g., cylindrical, structure having a proximal and distal end. The proximal end may include a connector element, e.g., an IS-1 connector, for connecting to a control unit, e.g., present in a “can” or analogous device. The lead may include one or more lumens, e.g., for use with a guidewire, for housing one or more conductive elements, e.g., wires, etc. The distal end may include a variety of different features as desired, e.g., a securing means, etc.

In certain embodiments of the subject systems, one or more sets of electrode assemblies or satellites as described above are electrically coupled to at least one elongated conductive member, e.g., an elongated conductive member present in a lead, such as a cardiovascular lead. For example, two or more assemblies are coupled to a common at least one electrical conductor, i.e., to the same at least one electrical conductor. In certain embodiments, the elongated conductive member is part of a multiplex lead. Multiplex lead structures may include 2 or more satellites, such as 3 or more, 4 or more, 5 or more, 10 or more, 15 or more, 20 or more, etc. as desired, where in certain embodiments multiplex leads have a fewer number of conductive members than satellites. In certain embodiments, the multiplex leads include 3 or less wires, such as only 2 wires or only 1 wire. Multiplex lead structures of interest include those described in application Ser. No. 10/734,490 titled “Method and System for Monitoring and Treating Hemodynamic Parameters” filed on Dec. 11, 2003; PCT/US2005/031559 titled “Methods and Apparatus for Tissue Activation and Monitoring,” filed on Sep. 1, 2006; PCT/US2005/46811 titled “Implantable Addressable Segmented Electrodes” filed on Dec. 22, 2005; PCT/US2005/46815 titled “Implantable Hermetically Sealed Structures” filed on Dec. 22, 2005; and Ser. No. 11/734,617 titled “High Phrenic, Low Pacing Capture Threshold Pacing Devices and Methods” filed Apr. 12, 2007; the disclosures of the various multiplex lead structures of these applications being herein incorporated by reference. In some embodiments of the invention, the devices and systems may include onboard logic circuitry or a processor, e.g., present in a central control unit, such as a pacemaker can. In these embodiments, the central control unit may be electrically coupled to the lead by a connector, such as a proximal end IS-1 connection.

The shape of the distally distributed multi-electrode lead can be designed based on the application of the lead. This has been discussed in greater detail in Pending PCT Application Serial No. PCT/US2006/025648 filed on Jun. 30, 2006, the disclosure of which is herein incorporated by reference. For example, the lead can have an “S” shape, a spiral, an “L” shape, or a pigtail, to aid in anchoring the lead in the vein. The lead can taper down to a smaller diameter quickly, and maintain a uniform diameter in the portion with the electrodes. Alternatively, the lead can taper gradually from a larger to smaller diameter, so that each successive electrode is of a smaller diameter. This shape conforms to the natural shape of the vein, allowing more optimal contact for the electrodes.

The configuration of the electrodes in the distally distributed multi-electrode lead can also be designed to accommodate specific applications. There can be multiple ring electrodes placed at different locations along the distal portion of the lead. These can be hard-wired back to the control chip, located more proximal on the lead. Each of these electrodes can be individually activated by the control chip. In some embodiments, the individually addressable electrodes in the distally distributed satellite electrode located in the smaller diameter distal portion of the lead can be circumferentially aligned, allowing selective activation of electrodes facing in different directions. In some embodiments, two or more individually addressable electrodes in the distally distributed satellite electrode are arranged circumferentially around the implantable elongated flexible structure, or lead. Further, the circumferentially arranged individually addressable electrodes in the distal portion can be configured with a mechanism that allows them to expand once they are placed in the optimal vein location.

The distal electrodes can be ring electrodes, and encompass the entire circumference of the lead. They can also be made with another desirable cross section profile, such as rectangular, square, triangular, oval, etc. Since they do not surround a control chip, the electrodes can conform to a smaller diameter. They can be individually hard-wired back to the control chip, and can be individually activated.

In another embodiment, the electrodes are exposed on only a partial circumference of the lead, producing a more focused signal. With the electrodes exposed on one side, the lead can selectively pace heart tissue without unwanted tissue stimulation, e.g. stimulation of the phrenic nerve. The benefits and methods for this are discussed in greater detail in U.S. Provisional Application Ser. No. 60/807,289, filed on Jul. 13, 2006, the disclosure of which is herein incorporated by reference.

The spacing of the electrodes on the distal portion can be adjusted to produce a more focused conduction path for the pacing signal, minimizing the capture of undesired tissue. In one embodiment, a group of two or more electrodes can be placed close together to produce a shorter conduction path. Alternatively, they can be spaced evenly apart, to allow for the widest possible range of tissue to be captured. The electrodes can be placed from about 0:05 cm to about 3 cm apart, more specifically from about 0.2 cm to about 1 cm apart, most specifically about 0.5 cm apart.

In another embodiment of the distally distributed multi-electrode lead, there can be more than one control chip used to control the electrodes on the smaller diameter distal portion of the lead. The limiting factor in how many electrodes can be placed in the distal region is the number of wires that must be included and hard-wired back to the one or more chips in the proximal region. With hard-wired electrodes, in order to preserve the smaller diameter, there can be about 1 to about 12 distal electrodes, more specifically about 3 to about 6 distal electrodes, most specifically about 4 distal electrodes.

In another embodiment of the distally distributed multi-electrode lead, a plurality of electrodes is placed circumferential to the lead axis on the smaller diameter distal portion. They are each hard wired separately to a control chip located proximally. This adds the benefit of directional selectivity. It provides the advantage of being able to select the electrodes that are facing the myocardium, allowing more focused activation of the heart, with a lower pacing threshold, better powering, and avoidance of phrenic nerve capture or stimulation of other undesired tissue.

Another embodiment includes a plurality of individually addressable electrodes arranged circumferentially to the lead axis on the smaller diameter distal portion, and in this embodiment, the distal electrodes are expandable. Once the circumferentially arranged electrodes are in place in the desired location in the vein, the electrodes can expand until they come in contact with the wall of the vein, forming a bubble, or mushroom shape configuration. This allows the lead to take advantage of a smaller diameter tip for entry into the vein, and once the electrodes are expanded provides better contact with the vein wall and improved anchoring, while maintaining directional selectivity of the electrodes. The lead can be molded to be naturally in the expanded state, with struts that are forced down during delivery of the lead.

Many mechanisms can be used to control the expansion of the electrodes in the distally distributed multi-electrode lead. In one embodiment, a stylet can be placed through the distal region, which can elongate the area of the lead containing the distal electrodes, which causes them to collapse. The stylet can then be locked with a turnkey mechanism. Once the lead is delivered in the desired location, the stylet can be unlocked and removed, allowing the electrodes to expand. If the lead needs to be removed, the stylet can be replaced and locked in place, collapsing the electrodes for easier removal.

In another embodiment of the distally distributed multi-electrode lead, the shape memory characteristics of NiTi can be used to provide a collapsed state at room temperature. Once the lead is placed in the body and reaches internal body temperature, the NiTi reshapes itself into an expanded state. If the lead needs to be removed, a cooling agent can be injected through the lead until the NiTi returns to the collapsed state.

In a further embodiment of the distally distributed multi-electrode lead, a plurality of expanding electrode sets can be placed on the distal portion of the lead. To reduce the diameter required by the conducting wires, the electrodes in each of the expanding sets can all be hard-wired to a single wire, so that only one wire is needed for each expanding electrode set.

In an embodiment of the distally distributed multi-electrode lead, the distal portion of the lead can take on a curved “S” shape. This aids in anchoring the lead, and in orienting the electrodes toward the vein surface. The lead can be delivered in a straight configuration with a guiding catheter or other delivery tool. When the delivery tool is removed, the distal tip takes on a curved “S” shape. In other embodiments, the distal portion can be configured in other shapes, such as a spiral, an “L”, or a pigtail.

In another embodiment of the distally distributed multi-electrode lead, the entire length of the smaller diameter distal portion of the lead is configured in a sinusoidal “S” shape, and the electrodes are located along the curves. The electrodes can be placed at the apex of the bends, which maximizes the distance between electrodes, and therefore the surface area covered by them. Alternatively, the electrodes can be placed off the apex of the curves. This configuration has the advantage of making it easier to remove the delivery tools because an electrode located at the apex may introduce increased bending stiffness coincident with the bends of the lead.

In another embodiment of the distally distributed multi-electrode lead, the lead can be in a sinusoidal shape with progressively smaller bends in the distal direction. This helps the lead conform to the tapering diameter of the vein, and also makes it easier to remove the delivery tools from the lead. In yet other embodiments, the entire length of the lead, including the proximal satellites, can be configured in a shape, such as a sinusoidal “S” curve, a spiral, an “L”, or a pigtail.

The lead body can be made of silicone or polyurethane. The electrodes may be made of platinum-iridium and coated with titanium-nitride or iridium-oxide, or any other material suitable for use in a human body. The conductor cable may be made of MP35N, or any other material with high fatigue strength. Many of the electrical connections can be made using a laser welding process. Standard molding techniques may be used to assemble the lead shape.

In other embodiments of the distally distributed multi-electrode leads described above, the lead can comprise a combination of any of the above mentioned elements. For example, the lead can contain at least one multi-directional electrode set, with one or more ring electrodes, and be configured in a sinusoidal shape.

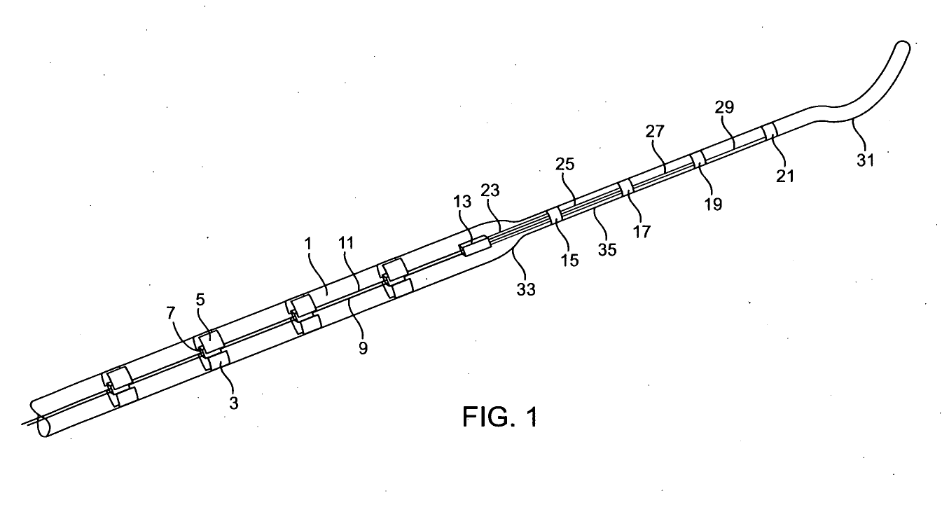

FIG. 1 illustrates an embodiment of the distally distributed multi-electrode lead 1 with segmented electrode satellites 3 located on the proximal end. Each of these segmented electrode satellites 3 consists of four electrically isolated electrodes 5 situated circumferentially around a control chip 7. Each control chip 7 is connected to two conducting wires 9 and 11, which communicate with the chips through a multiplexed two-wire bus system. The conducting wires 9 and 11 connect on the proximal end to the pacemaker can (not pictured).

The control chip receives instructions from and send information back to the pacemaker can via conducting wires 9, 11. When instructed to do so, control chip 7 can control electrodes 5. The chip 7 can control whether each electrode 5 acts as a cathode, an anode, or is turned off, as well as vary the voltage applied. The chip 7 can also configure the electrode 5 for pacing, sensing, or another function.

Located distally to the segmented electrode satellites is a hermetically sealed chip 13 which is very similar in design and capability to chip 7. Chip 13 is also connected to the two conducting wires 9 and 11. The diameter of the tapered portion of the lead 33 tapers between Chip 13 and electrode 15 to a diameter 35 which remains constant through the remaining length of the lead. Chip 13 is hard wired to ring electrodes 15, 17, 19, and 21. Wire 23 connects chip 13 to electrode 15, wire 25 connects chip 13 to electrode 17, wire 27 connects chip 13 to electrode 19, and wire 29 connects chip 13 to electrode 21. Distal tip 31 has a curved “S” shape to aid in anchoring the lead in the vein.

The lead 1 may be introduced to the body through a subclavian vein. A guide catheter may be used to find the coronary sinus in the heart. A guide wire is placed through the guide catheter into the coronary sinus, and navigated to one of several left ventricular coronary veins. With the guide wire in place, the lead is slid over the guide wire into one of the left ventricular coronary veins. The lead may be delivered in a straight configuration with the help of the guide wire, and can take on the curvature at the distal tip once the guide wire is removed. The lead may also be used elsewhere in the vasculature anatomy where the distal reach allowed by the narrow distal portion, combined with the selectivity of the electrodes throughout the lead would be an asset.

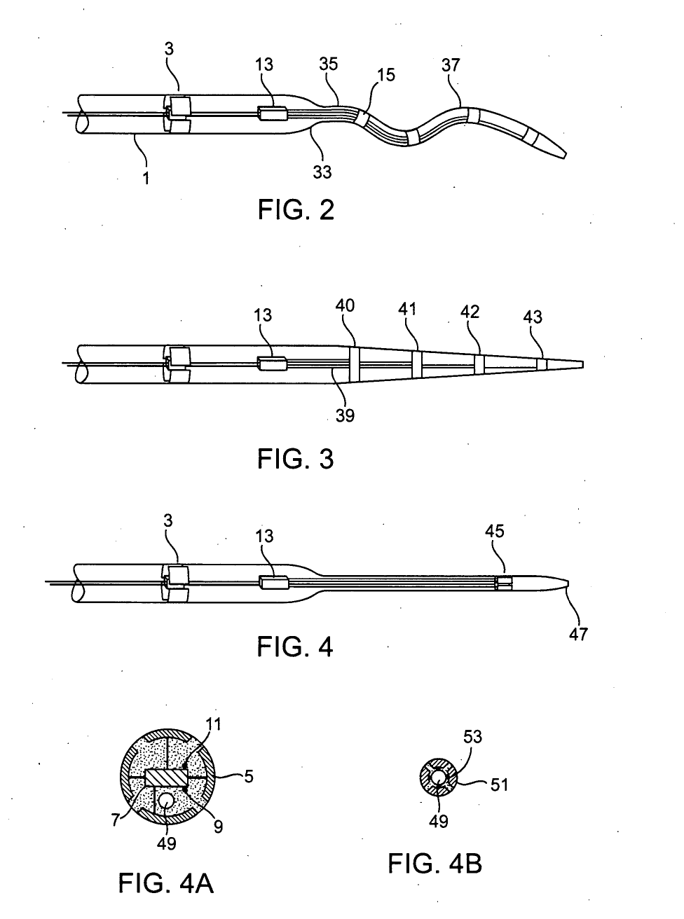

In another embodiment of the distally distributed multi-electrode lead, FIG. 2 shows a similar configuration with segmented electrode satellite 3 located on lead 1, with hermetically sealed chip 13 located distally to it, and electrodes 15 located distally to the chip, following a taper 33 to a uniform diameter 35. Following taper 33, the remaining length of the lead is configured in sinusoidal “S” shape 37. The lead 1 is delivered in a straight configuration with the use of guiding catheters or other delivery tools. When the delivery tools are removed, the lead expands to assume the sinusoidal configuration. With the lead in the sinusoidal configuration, it can provide better anchoring in the vein, along with improved electrical contact with the surrounding tissue.

FIG. 3 shows an embodiment of the invention in which the lead gradually tapers from the hermetically sealed chip 13 to the tip. This is facilitated by the fact that progressively fewer wires 39 are needed at the distal portions of the lead. The distal electrodes 40-43 are progressively smaller at the more distal locations. This configuration conforms to the natural shape of the vein at the distal portion, providing better contact between the electrodes and the surrounding tissue. If there is a lot of space between the electrode and the vein wall, the contact is not optimal. The gradually tapered configuration also provides a better fit between the distal portion of the lead and the vein, helping to anchor the lead.

Another embodiment, shown in FIG. 4, shows segmented satellite electrode structure 3, followed by stand-alone hermetically sealed chip 13, which is hard-wired to electrodes 45, which are arranged circumferentially around the lead axis on the smaller diameter distal portion of the lead. The lead then ends with a final taper 47, which aids the lead in moving through the vein. Having electrodes 45 arranged circumferentially provides the advantage of selectable directionality. It is an advantage to be able to selectively excite the electrodes that are facing the myocardium, to allow for more focused activation of the heart, with a lower activation threshold, better powering, and avoidance of phrenic nerve capture or stimulation of other undesired tissue. FIG. 4A shows the cross-section at segmented satellite electrode 3 from FIG. 4. Electrodes 5 are connected to hermetically sealed chip 7, and are circumferentially arranged around it. Wires 9 and 11 run along most of the length of the lead and are used to address each of the hermetically sealed chips. Guide wire lumen 49 is where the guide wire is placed while positioning the lead.

FIG. 4B shows the cross-section at segmented electrodes 45 from FIG. 4. Electrodes 51 are arranged circumferentially around the lead axis, and the connecting wires from the hermetically sealed chip are laser welded at points 53. The guide wire runs through guide wire lumen 49 while placing the lead. Note that there is no hermetically sealed chip in the center of this cross-section, allowing the diameter to be significantly smaller. This allows the distal portion of the lead to reach farther into the vein, while still providing directional selectivity.

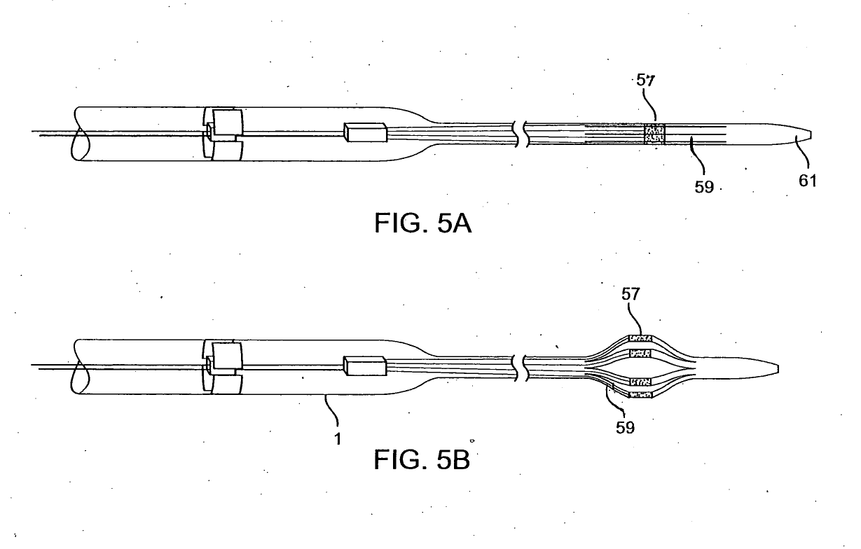

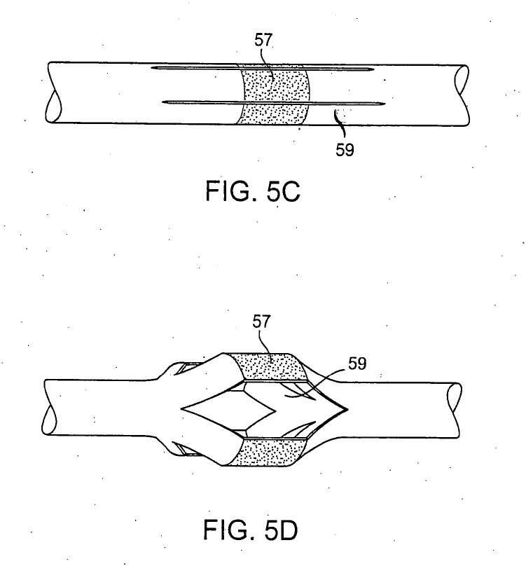

In another embodiment of the invention, the distal segmented electrode set can be expanded once it is placed in the vein to provide better contact between the electrodes and surrounding tissue, and to assist in anchoring the lead in the vein. FIG. 5A shows distal segmented electrodes 57 in their collapsed state. In this embodiment, each electrode 57 is attached to a strut 59 and is expanded in the natural state, and collapsed when they are stretched out. The struts 59 are locked in the collapsed state using a stylet (not shown) and turnkey mechanism in the distal tip of the lead. The stylet is inserted in the lead, prior to insertion into the body, to collapse the struts, then locked into place using the turnkey mechanism. With the electrodes 57 collapsed, the lead can be more easily positioned into the vein. The tip 61 extends past the electrode set, and has a tapered tip to aid in pushing through the vein.

FIG. 5B shows the segmented electrodes 57 from FIG. 5A in their expanded state. Once the lead 1 is in place, the stylet is pulled back or removed, allowing the struts 59 to push the electrodes 57 to the vein wall. It is important for the struts 59 to have enough force to push the electrodes to the vein wall, but keep the pressure low enough not to damage the vein. The expanding electrodes provide more optimal contact with the surrounding tissue and also help to anchor the lead in the vein, while still providing directional selectivity. If the lead needs to be removed, the stylets can be replaced to collapse the struts and allow for easier removal.

FIG. 5C is a closer view of the expandable distal segmented electrodes 57 in their collapsed state, with stylet (not shown) locking the struts 59 in the collapsed state.

FIG. 5D is a closer view of the expandable distal segmented electrodes 57 in their expanded state, with the stylet removed, and struts 59 pushing the electrodes 57 to the vein wall.

The leads may further include a variety of different effector elements, which elements may employ the satellites or structures distinct from the satellites. The effectors may be intended for collecting data, such as but not limited to pressure data, volume data, dimension data, temperature data, oxygen or carbon dioxide concentration data, hematocrit data, electrical conductivity data, electrical potential data, pH data, chemical data, blood flow rate data, thermal conductivity data, optical property data, cross-sectional area data, viscosity data, radiation data and the like. As such, the effectors may be sensors, e.g., temperature sensors, accelerometers, ultrasound transmitters or receivers, voltage sensors, potential sensors, current sensors, etc. Alternatively, the effectors may be intended for actuation or intervention, such as providing an electrical current or voltage, setting an electrical potential, heating a substance or area, inducing a pressure change, releasing or capturing a material or substance, emitting light, emitting sonic or ultrasound energy, emitting radiation and the like.

Effectors of interest include, but are not limited to, those effectors described in the following applications by at least some of the inventors of the present application: U.S. patent application Ser. No. 10/734,490 published as 20040193021 titled: “Method And System For Monitoring And Treating Hemodynamic Parameters”; U.S. patent application Ser. No. 11/219,305 published as 20060058588 titled: “Methods And Apparatus For Tissue Activation And Monitoring”; International Application No. PCT/US2005/046815 titled: “Implantable Addressable Segmented Electrodes”; U.S. patent application Ser. No. 11/324,196 titled “Implantable Accelerometer-Based Cardiac Wall Position Detector”; U.S. patent application Ser. No. 10/764,429, entitled “Method and Apparatus for Enhancing Cardiac Pacing,” U.S. patent application Ser. No. 10/764,127, entitled “Methods and Systems for Measuring Cardiac Parameters,” U.S. patent application Ser. No. 10/764,125, entitled “Method and System for Remote Hemodynamic Monitoring”; International Application No. PCT/US2005/046815 titled: “Implantable Hermetically Sealed Structures”; U.S. application Ser. No. 11/368,259 titled: “Fiberoptic Tissue Motion Sensor”; International Application No. PCT/US2004/041430 titled: “Implantable Pressure Sensors”; U.S. patent application Ser. No. 11/249,152 entitled “Implantable Doppler Tomography System,” and claiming priority to: U.S. Provisional Patent Application No. 60/617,618; International Application Serial No. PCT/USUS05/39535 titled “Cardiac Motion Characterization by Strain Gauge”. These applications are incorporated in their entirety by reference herein.

Implantable Pulse Generators

Embodiments of the invention further include implantable pulse generators. Implantable pulse generators may include: a housing which includes a power source and an electrical stimulus control element; one or more implantable elongated flexible structures, or vascular leads, as described above, e.g., 2 or more vascular leads, where each lead is coupled to the control element in the housing via a suitable connector, e.g., an IS-1 connector. In certain embodiments, the implantable pulse generators are ones that are employed for cardiovascular applications, e.g., pacing applications, cardiac resynchronization therapy applications, etc. As such, in certain embodiments the control element is configured to operate the pulse generator in a manner so that it operates as a pacemaker, e.g., by having an appropriate control algorithm recorded onto a computer readable medium of a processor of the control element. In certain embodiments the control element is configured to operate the pulse generator in a manner so that it operates as a cardiac resynchronization therapy device, e.g., by having an appropriate control algorithm recorded onto a computer readable medium of a processor of the control element.

Summarizing aspects of the above description, in using the implantable pulse generators of the invention, such methods include implanting an implantable pulse generator e.g., as described above, into a patient; and the implanted pulse generator, e.g., to deliver electrical stimulation to the tissue (e.g. cardiac tissue) of the patient, to pace the heart of the patient, to perform cardiac resynchronization therapy in the patient, etc. The description of the present invention is provided herein in certain instances with reference to a subject or patient. As used herein, the terms “subject” and “patient” refer to a living entity such as an animal. In certain embodiments, the animals are “mammals” or “mammalian,” where these terms are used broadly to describe organisms which are within the class mammalia, including the orders carnivore (e.g., dogs and cats), rodentia (e.g., mice, guinea pigs, and rats), lagomorpha (e.g. rabbits) and primates (e.g., humans, chimpanzees, and monkeys). In certain embodiments, the subjects, e.g., patients, are humans.

During operation, use of the implantable pulse generator may include activating at least one of the electrodes of the pulse generator to deliver electrical energy to the subject, where the activation may be selective, such as where the method includes first determining which of the electrodes of the pulse generator to activate and then activating the electrode. Methods of using an IPG, e.g., for pacing and CRT, are disclosed in Application Serial Nos.: PCT/US2005/031559 titled “Methods and Apparatus for Tissue Activation and Monitoring,” filed on Sep. 1, 2006; PCT/US2005/46811 titled “Implantable Addressable Segmented Electrodes” filed on Dec. 22, 2005; PCT/US2005/46815 titled “Implantable Hermetically Sealed Structures” filed on Dec. 22, 2005; and Ser. No. 11/734,617 titled “High Phrenic, Low Capture Threshold Pacing Devices and Methods,” filed Apr. 12, 2006; the disclosures of the various methods of operation of these applications being herein incorporated by reference and applicable for use of the present devices.

The devices and systems of the invention may find use in, methods of highly specific tissue stimulation, e.g., highly specific cardiac tissue stimulation. Where the tissue that is stimulated in the subject methods is cardiac tissue, embodiments of the methods of cardiac tissue stimulation may be characterized as high phrenic nerve capture threshold, low cardiac tissue capture threshold methods. In these embodiments, cardiac tissue is stimulated in a manner such that the capture threshold for the phrenic nerve is significantly higher than the capture threshold for the cardiac tissue, e.g., about 5 times or more higher, such about 10 times or more higher and including about 20 times more or higher. In certain embodiments, the capture of the phrenic nerve only occurs with activation energies of about 3 to about 18 volts or higher, such as about 10 to about 17 volts or higher, including about 15 volts or higher.

Where desired, the methods may include a step of obtaining phrenic nerve capture data and employing this data in the selective tissue stimulation. For example, a sensor can be employed to detect phrenic nerve capture, and the resultant data employed to set or more modify the cardiac stimulation parameters of focused cardiac stimulation. The sensor may be present in the same lead or a different lead from the cardiac stimulation lead. Any convenient sensor may be employed. The sensor could be an electrical sensor if it is on the diaphragm or near the phrenic nerve or it could be a motion sensor or a mechanical motion sensor on the lead. Examples of suitable sensors include pressure sensors, strain gauges, accelerometers, acoustic sensors, where the sensors can be orientated anywhere along the lead or independently on another lead placed on the diaphragm.

In certain embodiments, feedback regarding phrenic nerve capture or lack thereof is provided so that if one is automatically repositioning electrodes the box can have a feedback mechanism and the circuit can make sure that it does not choose an inappropriate electrode that would cause phrenic stimulation. In addition, during the initial programming of the device it could provide feedback that would be sub-threshold or tactile threshold for the clinician when he is observing the patient or possibly also for the patient.

In other embodiments, data regarding phrenic nerve capture, e.g., from distinct devices associated with the diaphragm, such as a diaphragm lead, can be employed. Any convenient method of communicating the data from the diaphragm specific lead to the controller of the pacing lead may be employed, such as an RF or other suitable communication protocol.

As such, the phrenic nerve capture device could be inside the cardiac stimulation lead or associated with a deminimus ASIC chip or it could be a separate packaged assembly inside the lead and not exposed.

One can evaluate for a correlation between pacing pulses and EMG signals around diaphragm or phrenic nerve signals.

Systems

Also provided are systems that include one more devices as described above. The systems of the invention may be viewed as systems for communicating information within the body of subject, e.g., human, where the systems include both a first implantable medical device, such as an IPG device described above, that includes a transceiver configured to transmit and/or receive a signal; and a second device comprising a transceiver configured to transmit and/or receive a signal. The second device may be a device that is inside the body, on a surface of the body or separate from the body during use.

Also provided are methods of using the systems of the invention. The methods of the invention generally include: providing a system of the invention, e.g., as described above, that includes first and second medical devices, one of which may be implantable; and transmitting a signal between the first and second devices. In certain embodiments, the transmitting step includes sending a signal from the first to said second device. In certain embodiments, the transmitting step includes sending a signal from the second device to said first device. The signal may transmitted in any convenient frequency, where in certain embodiments the frequency ranges from about 400 to about 405 MHz. The nature of the signal may vary greatly, and may include one or more data obtained from the patient, data obtained from the implanted device on device function, control information for the implanted device, power, etc.

Use of the systems may include visualization of data obtained with the devices. Some of the present inventors have developed a variety of display and software tools to coordinate multiple sources of sensor information which will be gathered by use of the inventive systems. Examples of these can be seen in international PCT application serial no. PCT/US2006/012246; the disclosure of which application, as well as the priority applications thereof are incorporated in their entirety by reference herein.

Kits

Also provided are kits that include the subject distally distributed multi-electrode leads, as part of one or more components of an implantable device or system, such as an implantable pulse generator, e.g., as reviewed above. In certain embodiments, the kits further include at least a control unit, e.g., in the form of a pacemaker can. In certain of these embodiments, the structure and control unit may be electrically coupled by an elongated conductive member. In certain embodiments, the electrode structure may be present in a lead, such as a cardiovascular lead.

In certain embodiments of the subject kits, the kits will further include instructions for using the subject devices or elements for obtaining the same (e.g., a website URL directing the user to a webpage which provides the instructions), where these instructions are typically printed on a substrate, which substrate may be one or more of: a package insert, the packaging, reagent containers and the like. In the subject kits, the one or more components are present in the same or different containers, as may be convenient or desirable.

It is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. An implantable elongated flexible structure comprising:
a proximal end;
a distal end; and
a distally distributed satellite electrode, wherein said distally distributed satellite electrode includes:
a processor; and
at least one individually addressable electrode coupled to said processor, wherein said at least one individually addressable electrode is located distal to said processor.

2. The implantable elongated flexible structure according to claim 1, wherein said distally distributed satellite electrode is a segmented electrode comprising two or more individually addressable electrodes located distal to said processor.

3. The implantable elongated flexible structure according to claim 2, wherein said distal end is tapered relative to said proximal end and said two or more individually addressable electrodes are positioned in said tapered end.

4. The implantable elongated flexible structure according to claim 3, wherein said processor of said distally distributed satellite electrode is not located in said tapered end.

5. The implantable elongated flexible structure according to claim 3, wherein said processor of said distally distributed satellite electrode is located in said tapered end.

6. The implantable elongated flexible structure according to claim 1, wherein said structure is a multi-electrode lead comprising at least one additional electrode satellite positioned proximal to said distally distributed satellite electrode.

7. The implantable elongated flexible structure according to claim 6, wherein said at least one additional electrode satellite is a segmented electrode.

8. The implantable elongated flexible structure according to claim 1, wherein said structure is a multiplex lead comprising 3 or less wires.

9. The implantable elongated flexible structure according to claim 8, wherein said lead includes only 2 wires.

10. The implantable elongated flexible structure according to claim 9, wherein said lead includes only 1 wire.

11. The implantable elongated flexible structure according to claim 1, wherein said structure is a vascular lead.

12. The implantable elongated flexible structure according to claim 11, wherein said vascular lead includes an IS-1 connector at said proximal end.

13. The implantable elongated flexible structure according to claim 1, wherein said processor and said at least one individually addressable electrode of said distally distributed satellite electrode are separated by a distance of 30 mm or less.

14. The implantable elongated flexible structure according to claim 13, wherein said processor and said at least one individually addressable electrode of said distally distributed satellite electrode are separated by a distance of 1 mm or more.

15. The implantable elongated flexible structure according to claim 1, wherein said distally distributed satellite electrode comprises two or more individually addressable electrodes that are arranged circumferentially around said structure.

16. The implantable elongated flexible structure according to claim 15, wherein said circumferentially arranged electrodes are expandable.

17. An implantable pulse generator comprising:
(a) a housing comprising a power source and an electrical stimulus control element; and
(b) an implantable elongated flexible structure according to claim 1.

18. The implantable pulse generator according to claim 17, wherein said control element is configured to operate said implantable pulse generator as a pacemaker.

19. The implantable pulse generator according to claim 18, wherein said control element is configured to operate said implantable pulse generator in a manner sufficient to achieve cardiac resynchronization.

20. A method comprising:
(a) implanting into a patient an implantable pulse generator comprising:
(i) a housing comprising a power source and an electrical stimulus control element; and
(ii) an implantable elongated flexible structure according to claim 1; and
(iii) delivering electrical stimulation to tissue of said patient.

21. The method according to claim 20, wherein said tissue is cardiac tissue.

22. The method according to claim 21, wherein said method is a method of cardiac pacing.

23. The method according to claim 22, wherein said method is a method of cardiac resynchronization therapy.

24. A kit comprising:
(a) a housing comprising a power source and an electrical stimulus control element; and
(b) an implantable elongated flexible structure according to claim 1.
